SEQUENCE LISTING (11 sequences)

SEQ ID NO: 1 - DNA, 8 bp, Artificial
  This is a modified Kozak′s sequence
ccgccatg

SEQ ID NO: 2 - DNA, 6 bp, Artificial
  This is a recombinant Eco R1 sequence
gaattc

SEQ ID NO: 3 - DNA, 713 bp, Porcine circovirus
cagctatgacgtatccaaggaggcgttaccgcagaagaagacaccgcccccgcagccatcttggccagatcctccgccgccgcccctggctcgtccacccccgccaccgctaccgttggagaaggaaaaatggcatcttcaacacccgcctctcccgcaccttcggatatactgtggagaaggaaaaatggcatcttcaacacccgcctctcccgcaccttcggatatactgtgacgactttgttcccccgggaggggggaccaacaaaatctctataccctttgaatactacagaataagaaaggttaaggttgaattctggccctgctcccccatcacccagggtgataggggagtgggctccactgctgttattctagatgataactttgtaacaaaggccacagccctaacctatgacccatatgtaaactactcctcccgccatacaatcccccaacccttctcctaccactcccgttacttcacacccaaacctgttcttgactccactattgattacttccaaccaaataacaaaaggaatcagctttggctgaggctacaaacctctagaaatgtggaccacgtaggcctcggcactgcgttcgaaaacagtaaatacgaccaggactacaatatccgtgtaaccatgtatgtacaattcagagaatttaatcttaaagaccccccacttaaaccctaaatgaat

SEQ ID NO: 4 - DNA, 713 bp, Porcine circovirus
ccgccatgacgtatccaaggaggcgttaccgcagaagaagacaccgcccccgcagccatcttggccagatcctccgccgccgcccctggctcgtccacccccgccaccgctaccgttggagaaggaaaaatggcatcttcaacacccgcctctcccgcaccttcggatatactgtcaaggctaccacagtcacaacgccctcctgggcggtggacatgatgagatttaatattgacgactttgttcccccgggaggggggaccaacaaaatctctataccctttgaatactacagaataagaaaggttaaggttgaattctggccctgctcccccatcacccagggtgataggggagtgggctccactgctgttattctagatgataactttgtaacaaaggccacagccctaacctatgacccatatgtaaactactcctcccgccatacaatcccccaacccttctcctaccactcccgttacttcacacccaaacctgttcttgactccactattgattacttccaaccaaataacaaaaggaatcagctttggctgaggctacaaacctctagaaatgtggaccacgtaggcctcggcactgcgttcgaaaacagtaaatacgaccaggactacaatatccgtgtaaccatgtatgtacaattcagagaatttaatcttaaagaccccccacttgaaccctaagaattc

SEQ ID NO: 5 - protein, 233 aa, Porcine circovirus
MTYPRRRYRRRRHRPRSHLGQILRRRPWLVHPRHRYRWRRKNGIFNTRLSRTFGYTVKATTVTTPSWAVDMMRFNIDDFVPPGGGTNKISIPFEYYRIRKVKVEFWPCSPITQGDRGVGSTAVILDDNFVTKATALTYDPYVNYSSRHTIPQPFSYHSRYFTPKPVLDSTIDYFQPNNKRNQLWLRLQTSRNVDHVGLGTAFENSKYDQDYNIRVTMYVQFREFNLKDPPLKP

SEQ ID NO: 6 - protein, 233 aa, Porcine circovirus
MTYPRRRYRRRRHRPRSHLGQILRRRPWLVHPRHRYRWRRKNGIFNTRLSRTFGYTVKATTVTTPSWAVDMMRFNIDDFVPPGGGTNKISIPFEYYRIRKVKVEFWPCSPITQGDRGVGSTAVILDDNFVTKATALTYDPYVNYSSRHTIPQPFSYHSRYFTPKPVLDSTIDYFQPNNKRNQLWLRLQTSRNVDHVGLGTAFENSKYDQDYNIRVTMYVQFREFNLKDPPLEP

SEQ ID NO: 7 - DNA, 756 bp, Artificial
  This sequence is from porcine circovirus type 2, open reading frame 2, together with a portion from the pGEM T-easy vector.
gcggccgcgggaattcgatccgccatgacgtatccaaggaggcgttaccgcagaagaagacaccgcccccgcagccatcttggccagatcctccgccgccgcccctggctcgtccacccccgccaccgctaccgttggagaaggaaaaatggcatcttcaacacccgcctctcccgcaccttcggatatactgtcaaggctaccacagtcacaacgccctcctgggcggtggacatgatgagatttaatattgacgactttgttcccccgggaggggggaccaacaaaatctctataccctttgaatactacagaataagaaaggttaaggttgaattctggccctgctcccccatcacccagggtgataggggagtgggctccactgctgttattctagatgataactttgtaacaaaggccacagccctaacctatgacccatatgtaaactactcctcccgccatacaatcccccaacccttctcctaccactcccgttacttcacacccaaacctgttcttgactccactattgattacttccaaccaaataacaaaaggaatcagctttggctgaggctacaaacctctagaaatgtggaccacgtaggcctcggcactgcgttcgaaaacagtaaatacgaccaggactacaatatccgtgtaaccatgtatgtacaattcagagaatttaatcttaaagaccccccacttgaaccctaagaattctatcactagtgaattcgcggccgc

SEQ ID NO: 8 - DNA, 10387 bp, Artificial
  This is the porcine circovirus type 2, ORF2 construct, which includes baculovirus and pGEM T-easy coding sequences.
aagctttactcgtaaagcgagttgaaggatcatatttagttgcgtttatgagataagattgaaagcacgtgtaaaatgtttcccgcgcgttggcacaactatttacaatgcggccaagttataaaagattctaatctgatatgttttaaaacacctttgcggcccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgatatcatggagataattaaaatgataaccatctcgcaaataaataagtattttactgttttcgtaacagttttgtaataaaaaaacctataaatattccggattattcataccgtcccaccatcgggcgcggatcagatctgcagcggccgcgggaattcgatccgccatgacgtatccaaggaggcgttaccgcagaagaagacaccgcccccgcagccatcttggccagatcctccgccgccgcccctggctcgtccacccccgccaccgctaccgttggagaaggaaaaatggcatcttcaacacccgcctctcccgcaccttcggatatactgtcaaggctaccacagtcacaacgccctcctgggcggtggacatgatgagatttaatattgacgactttgttcccccgggaggggggaccaacaaaatctctataccctttgaatactacagaataagaaaggttaaggttgaattctggccctgctcccccatcacccagggtgataggggagtgggctccactgctgttattctagatgataactttgtaacaaaggccacagccctaacctatgacccatatgtaaactactcctcccgccatacaatcccccaacccttctcctaccactcccgttacttcacacccaaacctgttcttgactccactattgattacttccaaccaaataacaaaaggaatcagctttggctgaggctacaaacctctagaaatgtggaccacgtaggcctcggcactgcgttcgaaaacagtaaatacgaccaggactacaatatccgtgtaaccatgtatgtacaattcagagaatttaatcttaaagaccccccacttgaaccctaagaattctatcactagtgaattcgcggccgccggccgctccagaattctagaaggtacccgggatcctttcctgggacccggcaagaaccaaaaactcactctcttcaaggaaatccgtaatgttaaacccgacacgatgaagcttgtcgttggatggaaaggaaaagagttctacagggaaacttggacccgcttcatggaagacagcttccccattgttaacgaccaagaagtgatggatgttttccttgttgtcaacatgcgtcccactagacccaaccgttgttacaaattcctggcccaacacgctctgcgttgcgaccccgactatgtacctcatgacgtgattaggatcgtcgagccttcatgggtgggcagcaacaacgagtaccgcatcagcctggctaagaagggcggcggctgcccaataatgaaccttcactctgagtacaccaactcgttcgaacagttcatcgatcgtgtcatctgggagaacttctacaagcccatcgtttacatcggtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcgccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgcggcgtgttaacatacaagttgctaacgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcc

SEQ ID NO: 9 - protein, 20 aa, Porcine circovirus
SYPRRRYRRRRHHPPSHLGQ

SEQ ID NO: 10 - protein, 19 aa, Porcine circovirus
PRHHYRPRRKNGIFNTTLS

SEQ ID NO: 11 - protein, 233 aa, Artificial
  This is an amino acid sequence for porcine circovirus type 2, open reading frame 2.
MTYPRRRYRRRRHRPRSHLGQILRRRPWLVHPRHRYRWRRKNGIFNTRLSRTFGYTVKATTVRTPSWAVDMMRFNIDDFVPPGGGTNKISIPFEYYRIKKVKVEFWPCSPITQGDRGVGSTAVILDDNFVTKATALTYDPYVNYSSRHTIPQPFSYHSRYFTPKPVLDSTIDYFQPNNKRNQLWLRLQTSRNVDHVGLGTAFENSIYDQDYNIRVTMYVQFREFNLKDPPLKP

(19) United States
(12) Patent Application Publication
(10) Pub. No.: US 2014/0348874 A1
(43) Pub. Date: Nov. 27, 2014

(54) METHOD FOR THE REDUCTION OF PCV-2 IN A HERD OF SWINE

(71) Applicants: Joaquim SEGALES, Vic (Barcelona) (ES); Marina SIBILA, Naras (Barcelona) (ES); Gerardo BLANCO ABILLA, Lleida (ES)

(72) Inventors: Joaquim SEGALES, Vic (Barcelona) (ES); Marina SIBILA, Naras (Barcelona) (ES); Gerardo BLANCO ABILLA, Lleida (ES)

(73) Assignee: BOEHRINGER INGELHEIM ESPANA, S.A., Sant Cugat del Valles (Barcelona) (ES)

(21) Appl. No.: 14/281,973

(22) Filed: May 20, 2014

(30) Foreign Application Priority Data
May 22, 2013 (EP) 13168639.6

(51) Int. Cl. A61K 39/12; C12N 7/00

(52) U.S. Cl. CPC A61K 39/12; C12N 7/00; C12N 2750/10034
USPC 424/204.1

(57) ABSTRACT

The present invention relates to a PCV2 ORF2 protein for use in a method for the reduction of the viral load, for the reduction of the infection quota and/or the reduction of the infection pressure in a herd of swine caused by an infection with PCV2. In particular, the present invention relates to any method described above, wherein said PCV2 ORF2 protein is to be administered at regular intervals to female animals of a herd and to piglets.

### SEQUENCE LISTING

This application contains a sequence listing in accordance with 37 C.F.R. 1.821-1.825. The sequence listing accompanying this application is hereby incorporated by reference in its entirety.

### BACKGROUND

1. Field of Invention

The present invention relates to a PCV2 ORF2 protein for use in a method for the reduction of the viral load, for the reduction of the infection quota and/or the reduction of the infection pressure in a herd of swine caused by an infection with PCV2. In particular, the present invention relates to any method described above, wherein said PCV2 ORF2 protein is to be administered at regular intervals to female animals of a herd and to piglets.

2. Description of the Prior Art

Porcine circovirus type 2 (PCV2) is a small (17-22 nm in diameter), icosahedral, non-enveloped DNA virus, which contains a single-stranded circular genome. PCV2 shares approximately 80% sequence identity with porcine circovirus type 1 (PCV-1). However, in contrast with PCV-1, which is generally non-virulent, swine infected with PCV2 exhibit a syndrome commonly referred to as Post-weaning Multisystemic Wasting Syndrome (PMWS). PMWS is clinically characterized by wasting, paleness of the skin, unthriftiness, respiratory distress, diarrhea, icterus, and jaundice. In some affected swine, a combination of all symptoms will be apparent while other swine will only have one or two of these symptoms. During necropsy, microscopic and macroscopic lesions also appear on multiple tissues and organs, with lymphoid organs being the most common site for lesions. A strong correlation has been observed between the amount of PCV2 nucleic acid or antigen and the severity of microscopic lymphoid lesions. Mortality rates for swine infected with PCV2 can approach 80%. In addition to PMWS, PCV2 has been associated with several other infections including pseudorabies, porcine reproductive and respiratory syndrome (PRRS), Glasser's disease, streptococcal meningitis, salmonellosis, post-weaning colibacillosis, dietetic hepatosis, and suppurative bronchopneumonia.

Several vaccines are available to reduce the impact of PCV2 infections in pigs. U.S. Pat. No. 6,703,023 provides a DNA-based vaccine for the prophylaxis of pigs against PMWS. In WO 03/049703, production of a live chimeric vaccine is described, comprising the non-pathogenic PCV1 virus in which, however, the ORF2 protein is replaced by the ORF2 protein of the pathogenic PCV-2. WO 99/18214 and WO 99/29717 have provided several PCV-2 strains and procedures for the preparation of a killed PCV2 vaccine, in particular to be used for the vaccination of sow. Preparation of subunit vaccines have also been described in WO 99/18214 and WO 99/29717. An effective ORF2 based subunit vaccine has been reported in WO 06/072065 and in WO2011/028888. A further ORF-2 based subunit vaccine is described also in WO 07/28823.

Registered PCV2 vaccines are to be applied to growing piglets around 2-4 weeks of age, and one of them was originally licensed as an inactivated vaccine to be used in gilts and sows (Kekarainen et al., 2010). These vaccines have been demonstrated to be efficient to control PCV2 infection under clinically and subclinically conditions in several experimental and field studies (Fachinger et al., 2008; Fort et al., 2008, 2009; Opriessnig et al., 2009; Segalés et al., 2009; Pejsak et al., 2010). Efficacy of PCV2 vaccines in piglets is based on reduction of number of PCV2 infected pigs and PCV2 viral load in those pigs as well as to control post-weaning multisystemic wasting syndrome (PMWS) clinical signs.

It has been reported that PCV2 infection and/or low serological titers to PCV2 in sows at farrowing increased the overall mortality of their offspring to PMWS (Allan et al. 2002; Calsamiglia et al. 2007).

### DESCRIPTION OF THE FIGURES

FIG. 1 is a table illustrating the results from analyzed samples from the transversal groups of piglets; and:

FIG. 2 is a series of four graphs illustrating the percentage of PCR and ELISA positive pigs (mean OD (±SD) ELISA values) in the four transversally analyzed groups.

### SUMMARY OF THE INVENTION

The practice of the present invention will employ, unless otherwise indicated, conventional techniques of molecular biology, microbiology, recombinant DNA technology, protein chemistry and immunology, which are within the skill of the art. Such techniques are explained fully in the literature. See, e.g., Sambrook, Fritsch & Maniatis, Molecular Cloning: A Laboratory Manual, Vols. I, II and III, Second Edition (1989); DNA Cloning, Vols. I and II (D. N. Glover ed. 1985); Oligonucleotide Synthesis (M. J. Gait ed. 1984); Nucleic Acid Hybridization (B. D. Hames & S. J. Higgins eds. 1984); Animal Cell Culture (R. K. Freshney ed. 1986); Immobilized Cells and Enzymes (IRL press, 1986); Perbal, B., A Practical Guide to Molecular Cloning (1984); the series, Methods In Enzymology (S. Colowick and N. Kaplan eds., Academic Press, Inc.); Protein purification methods—a practical approach (E. L. V. Harris and S. Angal, eds., IRL Press at Oxford University Press); and Handbook of Experimental Immunology, Vols. I-IV (D. M. Weir and C. C. Blackwell eds., 1986, Blackwell Scientific Publications).

Before describing the present invention in detail, it is to be understood that this invention is not limited to particular DNA, polypeptide sequences or process parameters as such may, of course, vary. It is also to be understood that the terminology used herein is for the purpose of describing particular embodiments of the invention only, and is not intended to be limiting. It must be noted that, as used in this specification and the appended claims, the singular forms “a”, “an” and “the” include plural referents unless the content clearly dictates otherwise. Thus, for example, reference to “an antigen” includes a mixture of two or more antigens, reference to “an excipient” includes mixtures of two or more excipients, and the like.

### Definitions

The term “PCV2 ORF2 protein” as used herein refers to the capsid protein of porcine circovirus type 2 that is encoded by the nucleic acid of open reading frame 2 or at least an antigenic portion thereof. In a preferred aspect, the PCV2 ORF2 protein is
i) a polypeptide comprising a sequence selected from the group consisting of SEQ ID NO: 5, SEQ ID NO: 6, SEQ ID NO: 9, SEQ ID NO: 10, and SEQ ID NO: 11;
ii) any polypeptide that is at least 90% homologous to the polypeptide of i);
iii) any immunogenic portion of the polypeptides of i) and/or ii);
iv) the immunogenic portion of iii), comprising at least 10 contiguous amino acids included in a sequence selected from the group consisting of SEQ ID NO: 5, SEQ ID NO: 6, SEQ ID No: 9, SEQ ID No: 10, and SEQ ID No: 11;
v) a polypeptide that is encoded by a DNA comprising the sequence of SEQ ID NO: 3 or SEQ ID NO: 4;
vi) any polypeptide that is encoded by a polynucleotide that is at least 80% homologous to the polynucleotide of v);
vii) any immunogenic portion of the polypeptides encoded by the polynucleotide of v) and/or vi);
viii) the immunogenic portion of vii), wherein polynucleotide coding for said immunogenic portion comprises at least 30 contiguous nucleotides included in the sequences of SEQ ID No: 3, or SEQ ID No: 4.

The term “antigen” or “antigenic portion” of a PCV2 ORF2 protein refers to a polypeptide that codes for at least one antigen of PCV2 ORF2 protein.

The term “antigen” as used herein refers to an amino acid sequence which elicits an immune response in a host. An “antigen” or an “antigen of PCV2 ORF2” as used herein, includes the full-length sequence of any PCV2 proteins, analogs thereof, or immunogenic portion or fragment thereof or immunogenic portion thereof.

The term “immunogenic fragment” refers to a fragment of a protein which includes one or more epitopes and thus elicits the immune response in a host. Such fragments can be identified using any number of epitope mapping techniques well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715. Similarly, conformational epitopes are readily identified by determining spatial conformation of amino acids such as by, e.g., x-ray crystallography and 2-dimensional nuclear magnetic resonance. See, e.g., Epitope Mapping Protocols, supra. Synthetic antigens are also included within the definition, for example, polyepitopes, flanking epitopes, and other recombinant or synthetically derived antigens. See, e.g., Bergmann et al. (1993) Eur. J. Immunol. 23:2777-2781; Bergmann et al. (1996), J. Immunol. 157:3242-3249; Suhrbier, A. (1997), Immunol. and Cell Biol. 75:402-408; Gardner et al., (1998) 12th World AIDS Conference, Geneva, Switzerland, Jun. 28-Jul. 3, 1998.

An “immune response” means, but is not limited to, the development in a host of a cellular and/or antibody-mediated immune response to an antigen, an immunogenic composition, or a vaccine of interest. Usually, an “immune response” includes but is not limited to one or more of the following effects: the production or activation of antibodies, B cells, helper T cells, suppressor T cells, and/or cytotoxic T cells, directed specifically to an antigen or antigens included in the composition or vaccine of interest. Preferably, the host will display either a therapeutic or a protective immunological (memory) response such that resistance to new infection will be enhanced and/or the clinical severity of the disease reduced. Such protection will be demonstrated by either a reduction in number or severity of, or lack of one or more of the symptoms associated with PCV2 infections, in delay of onset of viremia, in a reduced viral persistence, in a reduction of the overall viral load, and/or a reduction of viral excretion.

The term “immunological composition comprising a PCV2 ORF2 protein” as used herein refers to any pharmaceutical composition containing a PCV2 ORF2 protein, which composition can be used to prevent or treat a PCV2 infection-associated disease or condition in a subject. A preferred immunogenic composition can induce, stimulate or enhance the immune response against PCV2. The term thus encompasses both subunit immunogenic compositions, as described below, as well as compositions containing whole killed, or attenuated, and/or inactivated PCV2. The immunogenic composition comprising such PCV2 ORF2 protein is able to elicit an “immunological response” in the host of a cellular and/or antibody-mediated immune response to PCV2 ORF2 protein. Preferably, this immunogenic composition is capable to confer protective immunity against PCV2 infection and the clinical signs associated therewith. In some forms, immunogenic portions of PCV2 ORF2 protein are used as the antigenic component in the composition.

The term “subunit immunogenic composition” as used herein refers to a composition containing at least one immunogenic polypeptide or antigen of PCV2 ORF2 protein, but not all antigens, derived from or homologous to an antigen from PCV2. Such a composition is substantially free of intact PCV2. Thus, a subunit immunogenic composition is prepared from at least partially purified or fractionated (preferably substantially purified) immunogenic polypeptides from PCV2 that at least comprises PCV2 ORF2 protein, or recombinant analogs thereof. Most preferred are immunogenic subunit compositions, which comprise any of the PCV2 antigens provided in WO06/072065, which are all incorporated herein by reference in their entirety.

The term “an effective amount of PCV2 antigen” or “an effective amount of PCV2 ORF2 protein” as used herein means but is not limited to an amount of PCV2 ORF2 protein or any antigenic portion thereof, that elicits or is able to elicit an immune response in an animal, to which said effective amount of PCV2 antigen is administered. The amount that is effective depends on the ingredients of the vaccine and the schedule of administration. Typically, when an inactivated virus or a modified live virus preparation is used in the combination vaccine, an amount of the vaccine containing about 10^{2.0} to about 10^{9.0} TCID₅₀ (tissue culture infective dose 50% end point)/dose per dose, preferably about 10^{3.0} to about 10^{8.0} TCID₅₀ per dose, more preferably, about 10^{4.0} to about 10^{8.0} TCID₅₀ per dose is used. In particular, when modified live PCV2 is used in the vaccines, the recommended dose to be administered to the susceptible animal is preferably about 10^{3.0} TCID₅₀ to about 10^{6.0} TCID₅₀/dose and more preferably about 10^{4.0} TCID₅₀/dose to about 10^{5.0} TCID₅₀/dose. In general, the quantity of antigen will be between 0.2 and 5000 micrograms, and between 10^{2.0} and 10^{9.0} TCID₅₀, preferably between 10^{3.0} and 10^{6.0} TCID₅₀, more preferably between 10^{4.0} and 10^{5.0} TCID₅₀, when purified antigen is used.

Sub-unit vaccines comprising PCV2ORF2 protein are normally administered with an antigen inclusion level of at least 0.2 μg antigen per dose, preferably with about 0.2 to about 400 μg/dose, still more preferably with about 0.35 to about 200 μg/dose, even more preferably with about 0.7 to about 100 μg/dose, still more preferably with about 1.0 to about 80 μg/dose, still more preferably with about 1.5 to about 70 μg/dose, still more preferably with about 2.0 to about 60 μg/dose, even more preferably with about 3.0 to about 50 μg/dose, even more preferably with about 4.0 to about 40 μg/dose, still more preferably with about 4.0 to about 24 μg/dose.

The immunogenic composition as used herein also may include the PCV2 ORF2 protein as included in INGELVAC® CIRCOFLEX, (Boehringer Ingelheim Vetmedica Inc., St Joseph, Mo., USA), CIRCOVAC® (Merial SAS, Lyon, France), CircoVent (Intervet Inc., Millsboro, Del., USA), or SUVAXYN PCV-2 ONE DOSE® (Fort Dodge Animal Health, Kansas City, Kans., USA).

“Sequence Identity” as it is known in the art refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48: 1073 (1988), the teachings of which are incorporated herein by reference. Preferred methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12(1):387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S. et al., NCVI NLM NIH Bethesda, Md. 20894, Altschul, S. F. et al., J. Molec. Biol., 215:403-410 (1990), the teachings of which are incorporated herein by reference). These programs optimally align sequences using default gap weights in order to produce the highest level of sequence identity between the given and reference sequences. As an illustration, by a polynucleotide having a nucleotide sequence having at least, for example, 85%, preferably 90%, even more preferably 95% “sequence identity” to a reference nucleotide sequence, it is intended that the nucleotide sequence of the given polynucleotide is identical to the reference sequence except that the given polynucleotide sequence may include up to 15, preferably up to 10, even more preferably up to 5 point mutations per each 100 nucleotides of the reference nucleotide sequence. In other words, in a polynucleotide having a nucleotide sequence having at least 85%, preferably 90%, even more preferably 95% identity relative to the reference nucleotide sequence, up to 15%, preferably 10%, even more preferably 5% of the nucleotides in the reference sequence may be deleted or substituted with another nucleotide, or a number of nucleotides up to 15%, preferably 10%, even more preferably 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having a given amino acid sequence having at least, for example, 85%, preferably 90%, even more preferably 95% sequence identity to a reference amino acid sequence, it is intended that the given amino acid sequence of the polypeptide is identical to the reference sequence except that the given polypeptide sequence may include up to 15, preferably up to 10, even more preferably up to 5 amino acid alterations per each 100 amino acids of the reference amino acid sequence. In other words, to obtain a given polypeptide sequence having at least 85%, preferably 90%, even more preferably 95% sequence identity with a reference amino acid sequence, up to 15%, preferably up to 10%, even more preferably up to 5% of the amino acid residues in the reference sequence may be deleted or substituted with another amino acid, or a number of amino acids up to 15%, preferably up to 10%, even more preferably up to 5% of the total number of amino acid residues in the reference sequence may be inserted into the reference sequence. These alterations of the reference sequence may occur at the amino or the carboxy terminal positions of the reference amino acid sequence or anywhere between those terminal positions, interspersed either individually among residues in the reference sequence or in the one or more contiguous groups within the reference sequence. Preferably, residue positions which are not identical differ by conservative amino acid substitutions. However, conservative substitutions are not included as a match when determining sequence identity.

A “conservative substitution” refers to the substitution of an amino acid residue or nucleotide with another amino acid residue or nucleotide having similar characteristics or properties including size, hydrophobicity, etc., such that the overall functionality does not change significantly.

“Isolated” means altered “by the hand of man” from its natural state, i.e., if it occurs in nature, it has been changed or removed from its original environment, or both. For example, a polynucleotide or polypeptide naturally present in a living organism is not “isolated,” but the same polynucleotide or polypeptide separated from the coexisting materials of its natural state is “isolated”, as the term is employed herein.

The term “PCV2 positive” as used herein means, but is not limited to, an animal that comprises a detectable amount of PCV2 genome equivalents (=viral copies) in a sample (1 ml serum or 1 mg tissue). A detectable amount of PCV2 genome equivalents means that PCV2 genome equivalents can be detected by a polymerase chain reaction (PCR) assay. A sample is considered PCR positive if two independent samples provide a positive PCR result in such assay. Methods for quantification of PCV2 via a PCR assay are well known in the art. Preferably, the quantification of PCV2 genome equivalents was/is done by the method described in Brunborg et al., 2004; J. Virol Methods 122: 171-178. For amplification of PCV2, primers PCV2-84-1265U21 and PCV2-84-1319L21 were/are used. Such methods shall function as reference assay in any case of doubt.

The term “herd” as used herein refers a group of swine, in particular of all swine animals within the livestock of that farm.

The term “the reduction of the viral load” as used herein means that the number of PCR positive piglets within a herd of swine is reduced as compared to the number of PCR positive piglets in a herd of swine which herd of swine is not vaccinated according to the vaccination regime as described herein.

The term “PCR positive piglets” as used herein refer to piglets that have a detectable level of PCV2 virus genome equivalents in a sample of blood, preferably of one ml of blood. The quantitative PCR as described by Olvera, A et al., (J. Virol. Methods 117, 75-80, 2004) shall be used as a reference test to determine such positive negative piglets. A sample is considered to be “PCR negative” if no PCR signal is detected in two parallel blood samples.

The term “reduction of the infection quota” as used herein means, that the overall number of PCR positive swine in a herd of swine is reduced as compared to the number of PCR positive swine in a herd of swine which herd of swine is not vaccinated according to the vaccination regime as described herein.

The term “reduction of the infection pressure” as used herein means that the number of PCV2 infections in a herd of swine is reduced as compared to the number of PCV2 infections in a herd of swine which herd of swine is not vaccinated according to the vaccination regime as described herein.

The term “PCV2 infection” as used herein means a swine that shows (i) a detectable level of PCV2 virus genome equivalents in a sample of blood and/or (ii) clinical signs caused by a PCV2 infection.

The term “clinical signs caused by a PCV2 infection” as used herein refers to, but is not limited to, lung lesions, nasal shedding, cough, diarrhea, abnormal behavior, lethargy, lymphandenopathy, abnormal respirations, and combinations thereof.

The terms “reduction”, “reduced”, or “reduces” as used herein means a reduction of at least 10%, preferably of 20%, more preferably of 30%, more preferably of 40%, more preferably of 50%, more preferably of 60%, more preferably of 70%, more preferably of 80%, more preferably of 90%, even more preferably of 100% as compared to a respective control group.

The term “administration” as used herein means that the composition according to the invention may be applied intramuscularly, intranasally, intradermally, intravenously, intravascularly, intraarterially, intraperitoneally, subcutaneously, intradermally, intracutaneously, intracardially, intralobally, intramedullarly, intrapulmonarily, orally, intrathecally, intratracheally, or intravaginally. The composition preferably may be applied intramuscularly or intranasally. In an animal body, it can prove advantageous to apply the pharmaceutical compositions as described above via an intravenous injection or by direct injection into target tissues. For systemic application, the intravenous, intravascular, intramuscular, intranasal, intraarterial, intraperitoneal, oral, or intrathecal routes are preferred. A more local application can be effected subcutaneously, intradermally, intracutaneously, intracardially, intralobally, intramedullarly, intrapulmonarily or directly in or near the tissue to be treated (connective-, bone-, muscle-, nerve-, epithelial tissue). Depending on the desired duration and effectiveness of the treatment, the compositions according to the invention may be administered once or several times, also intermittently, for instance on a daily basis for several days, weeks or months, and in different dosages.

The term “regular intervals” as used herein means an interval of 3 to 12 months, preferably of 3 to 9 months, more preferably of 3 to 8 months, more preferably of 3 to 6 months, more preferably of 3 to 5 months, still more preferably of 3 or 4 months, more preferably of 4 months.

The term “female animal” as used herein means female swine of at least 5 months of age. A female swine preferably refers to a gilt. A “gilt” is a female swine selected for breeding normally of at least 5 to 6 months of age.

The term “piglets” as used herein means a newborn pig between 1 day of age and 5 months of age. Preferably, a piglet as used herein means a pig between 1 day of age and 4 months of age.

The term “swine” as used herein refers to swine, independently of age and gender. A swine include male and female piglets of each age as well as male and female swine of each age.

The term “adjuvants” as used herein, can include aluminum hydroxide and aluminum phosphate, saponins e.g., Quil A, QS-21 (Cambridge Biotech Inc., Cambridge Mass.), GPI-0100 (Galenica Pharmaceuticals, Inc., Birmingham, Ala.), water-in-oil emulsion, oil-in-water emulsion, water-in-oil-in-water emulsion. The emulsion can be based in particular on light liquid paraffin oil (European Pharmacopea type); isoprenoid oil such as squalane or squalene oil resulting from theoligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers are preferably nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the Pluronic products, especially L121. See Hunter et al., The Theory and Practical Application of Adjuvants (Ed. Stewart-Tull, D. E. S.). JohnWiley and Sons, NY, pp 51-94 (1995) and Todd et al., Vaccine 15:564-570 (1997). For example, it is possible to use the SPT emulsion described on page 147 of “Vaccine Design, The Subunit and Adjuvant Approach” edited by M. Powell and M. Newman, Plenum Press, 1995, and the emulsion MF59 described on page 183 of this same book.

A further instance of an adjuvant is a compound chosen from the polymers of acrylic or methacrylic acid and the copolymers of maleic anhydride and alkenyl derivative. Advantageous adjuvant compounds are the polymers of acrylic or methacrylic acid which are cross-linked, especially with polyalkenyl ethers of sugars or polyalcohols. These compounds are known by the term carbomer (Phameuropa Vol. 8, No. 2, June 1996). Persons skilled in the art can also refer to U.S. Pat. No. 2,909,462 which describes such acrylic polymers cross-linked with a polyhydroxylated compound having at least 3 hydroxyl groups, preferably not more than 8, the hydrogen atoms of at least three hydroxyls being replaced by unsaturated aliphatic radicals having at least 2 carbon atoms. The preferred radicals are those containing from 2 to 4 carbon atoms, e.g. vinyls, allyls and other ethylenically unsaturated groups. The unsaturated radicals may themselves contain other substituents, such as methyl. The products sold under the name Carbopol (BF Goodrich, Ohio, USA) are particularly appropriate. They are cross-linked with an allyl sucrose or with allyl pentaerythritol. Among them, there may be mentioned Carbopol 974P, 934P and 971P. Most preferred is the use of Carbopol, in particular the use of Carbopol 971P, preferably in amounts of about 500 μg to about 5 mg per dose, even more preferred in an amount of about 750 μg to about 2.5 mg per dose and most preferred in an amount of about 1 mg per dose.

Further suitable adjuvants include, but are not limited to, the RIBI adjuvant system (Ribi Inc.), Block co-polymer (CytRx, Atlanta Ga.), SAF-M (Chiron, Emeryville Calif.), monophosphoryl lipid A, Avridine lipid-amine adjuvant, heat-labile enterotoxin from E. coli (recombinant or otherwise), cholera toxin, IMS 1314, or muramyl dipeptide among many others.

Preferably, the adjuvant is added in an amount of about 100 μg to about 100 mg per dose. Even more preferably, the adjuvant is added in an amount of about 100 μg to about 10 mg per dose. Even more preferably, the adjuvant is added in an amount of about 500 μg to about 5 mg per dose. Even more preferably, the adjuvant is added in an amount of about 750 μg to about 2.5 mg per dose. Most preferably, the adjuvant is added in an amount of about 1 mg per dose.

The term “a pharmaceutical-acceptable carrier” as used herein includes any and all solvents, dispersion media, coatings, stabilizing agents, diluents, preservatives, antibacterial and antifungal agents, isotonic agents, adsorption delaying agents, and the like. Most preferably, the composition provided herewith contains PCV2 ORF2 protein recovered from the supernatant of in vitro cultured cells, wherein said cells were infected with a recombinant viral vector containing PCV2 ORF2 DNA and expressing PCV2 ORF2 protein, and wherein said cell culture was treated with about 2 to about 8 mM BEI, and more preferably with about 5 mM BEI to inactivate the viral vector, and an equivalent concentration of a neutralization agent, preferably sodium thiosulfate solution, to a final concentration of about 2 to about 8 mM, and more preferably of about 5 mM.

The tern “seroconversion”, “seroconverted” or “seroconvert” as used herein refers to a serological immune response as a result of an infection with wild-type PCV2 virus.

### DESCRIPTION OF THE INVENTION

In one aspect of the invention, PCV2 ORF2 protein or an immunological composition comprising a PCV2 ORF2 protein is provided for use in a method for the reduction of the viral load, for the reduction of the infection quota and/or for the reduction of the infection pressure in a herd of swine caused by an infection with PCV2, wherein said PCV2 ORF2 protein is to be administered at regular intervals to piglets and female animals of a herd.

Thus, in one aspect of the invention, PCV2 ORF2 protein or an immunological composition comprising a PCV2 ORF2 protein is provided for use in a method for the reduction of the viral load in a herd of swine caused by an infection with PCV2, wherein said PCV2 ORF2 protein is to be administered at regular intervals to piglets and female animals of a herd.

The viral load of PCV2 is in particular reduced in piglets of 12 to 16 weeks of age. Thus, in one aspect of the invention, PCV2 ORF2 protein or an immunological composition comprising a PCV2 ORF2 protein is provided for use in a method for the reduction of the viral load in piglets older than 12 weeks of age, preferably in piglets 12 to 20 weeks of age, in a herd of swine caused by an infection with PCV2, wherein said PCV2 ORF2 protein is to be administered at regular intervals to piglets and female animals of a herd.

In another aspect of the invention, PCV2 ORF2 protein or an immunological composition comprising a PCV2 ORF2 protein is provided for use in a method for the reduction of the infection quota in a herd of swine caused by an infection with PCV2, wherein said PCV2 ORF2 protein is to be administered at regular intervals to piglets and female animals of a herd.

In another aspect of the invention, PCV2 ORF2 protein or an immunological composition comprising a PCV2 ORF2 protein is provided for use in a method for the reduction of the infection pressure in a herd of swine caused by an infection with PCV2, wherein said PCV2 ORF2 protein is to be administered at regular intervals to piglets and female animals.

In another aspect of the invention, the PCV2 ORF2 protein or an immunological composition comprising a PCV2 ORF2 protein is provided for use according to any of the aspects herein, wherein said regular intervals are between 3 and 12 months, between 3 and 10 months, between 3 and 9 months, between 3 and 7 months, between 3 and 6 months, and between 3 and 4 months, with the most preferred range being between 3 and 4 months.

In another aspect of the invention, PCV2 ORF2 protein or an immunological composition comprising a PCV2 ORF2 protein is provided for use according to any of the aspects herein, wherein the regular interval is about 4 months.

In another aspect of the invention, PCV2 ORF2 protein or an immunological composition comprising a PCV2 ORF2 protein is provided for use according to any of the aspects herein, wherein the PCV2 ORF2 protein or immunological composition comprising a PCV2 ORF2 protein is to be administered during the whole lifetime of the female animals.

In another aspect of the invention, PCV2 ORF2 protein or an immunological composition comprising a PCV2 ORF2 protein is provided for use according to any of the aspects herein, wherein the PCV2 ORF2 protein or immunological composition comprising a PCV2 ORF2 protein is administered to the boars of the herd, preferably according to the same regular intervals as the female animals of the herd.

In another aspect of the invention, PCV2 ORF2 protein or an immunological composition comprising a PCV2 ORF2 protein is provided for use according to any of the aspects herein, wherein the PCV2 ORF2 protein or immunological composition comprising a PCV2 ORF2 protein is to be administered to 50% to 100% of the female swine animals in the herd, preferably to 60% to 100% of the female swine animals in the herd, even more preferably to 70% to 100% of the female swine animals in the herd, still more preferably to 80% to 100% of the female swine animals in the herd, and even more preferably administered to 90% to 100% of the female swine animals in the herd.

In another aspect of the invention, PCV2 ORF2 protein or an immunological composition comprising a PCV2 ORF2 protein is provided for use according to any of the aspects herein, wherein the PCV2 ORF2 protein or immunological composition comprising a PCV2 ORF2 protein is to be administered to all of the female swine animals in the herd.

In another aspect of the invention, PCV2 ORF2 protein or an immunological composition comprising a PCV2 ORF2 protein is provided for use according to any of the aspects herein, wherein said PCV2 ORF2 protein or immunological composition comprising a PCV2 ORF2 protein is to be administered one or more times to the piglets of the herd.

In another aspect of the invention, PCV2 ORF2 protein or an immunological composition comprising a PCV2 ORF2 protein is provided for use in a method for the reduction of the viral load, for the reduction of the infection quota and/or for the reduction of the infection pressure in a herd of swine caused by an infection with PCV2, wherein said PCV2 ORF2 protein or immunological composition comprising a PCV2 ORF2 protein is to be administered at regular intervals to female animals of a herd and to piglets, wherein said regular intervals for the female animals are between 3 and 12 months, between 3 and 10 months, between 3 and 9 months, between 3 and 7 months, between 3 and 6 months, and between 3 and 4 months, with the most preferred range being between 3 and 4 months, and wherein said PCV2 ORF2 protein or immunological composition comprising a PCV2 ORF2 protein is to be administered two or more times to the piglets within an interval between administrations of 2 to 6 weeks, an interval between administrations of 2 to 5 weeks, an interval between administrations of 2 to 4 weeks, or an interval between administrations of 2 to 3 weeks. According to one aspect, the viral load in piglets older than 12 weeks of age, preferably in piglets between 12 to 20 weeks of age, in a herd of swine is reduced.

In another aspect of the invention, PCV2 ORF2 protein or an immunological composition comprising a PCV2 ORF2 protein is provided for use in a method for the reduction of the viral load, wherein said PCV2 ORF2 protein or immunological composition comprising a PCV2 ORF2 protein is to be administered at regular intervals to female animals of a herd and to piglets, wherein said regular intervals are between 3 and 12 months, between 3 and 10 months, between 3 and 9 months, between 3 and 7 months, between 3 and 6 months, and between 3 and 4 months, with the most preferred range being between 3 and 4 months for the female animals and wherein said PCV2 ORF2 protein or immunological composition comprising a PCV2 ORF2 protein is to be administered two times to the piglets within an interval between administrations of 2 to 6 weeks, an interval between administrations of 2 to 5 weeks, an interval between administrations of 2 to 4 weeks, or an interval between administrations of 2 to 3 weeks. According to one aspect, the viral load in piglets older than 12 weeks of age, preferably in piglets between 12 to 20 weeks of age, in a herd of swine is reduced.

In another aspect of the invention, PCV2 ORF2 protein or an immunological composition comprising a PCV2 ORF2 protein is provided for use in a method for the reduction of the infection quota in a herd of swine caused by an infection with PCV2, wherein said PCV2 ORF2 protein or immunological composition comprising a PCV2 ORF2 protein is to be administered at regular intervals to female animals of a herd and to piglets, wherein said regular intervals are between 3 and 12 months, between 3 and 10 months, between 3 and 9 months, between 3 and 7 months, between 3 and 6 months, and between 3 and 4 months, with the most preferred range being between 3 and 4 months for the female animals, and wherein said PCV2 ORF2 protein is to be administered two or more times to the piglets within an interval between administrations of 2 to 6 weeks, an interval between administrations of 2 to 5 weeks, an interval between administrations of 2 to 4 weeks, or an interval between administrations of 2 to 3 weeks. According to one aspect, the viral load in piglets older than 12 weeks of age, preferably in piglets between 12 to 20 weeks of age, in a herd of swine is reduced.

In another aspect of the invention, PCV2 ORF2 protein or an immunological composition comprising a PCV2 ORF2 protein is provided for use in a method for the reduction of the infection pressure in a herd of swine caused by an infection with PCV2, wherein said PCV2 ORF2 protein or immunological composition comprising a PCV2 ORF2 protein is to be administered at regular intervals to female animals of a herd and to piglets, wherein said regular intervals for the female animals are between 3 and 12 months, between 3 and 10 months, between 3 and 9 months, between 3 and 7 months, between 3 and 6 months, and between 3 and 4 months, with the most preferred range being between 3 and 4 months, and wherein said PCV2 ORF2 protein or immunological composition comprising a PCV2 ORF2 protein is to be administered two times to the piglets within an interval between administrations of 2 to 6 weeks, an interval between administrations of 2 to 5 weeks, an interval between administrations of 2 to 4 weeks, or an interval between administrations of 2 to 3 weeks.

In another aspect of the invention, PCV2 ORF2 protein or an immunological composition comprising a PCV2 ORF2 protein is provided for use in a method for the reduction of the viral load, wherein said PCV2 ORF2 protein or immunological composition comprising a PCV2 ORF2 protein is to be administered at regular intervals to female animals of a herd and to piglets, wherein said regular intervals for the female animals are between 3 and 12 months, between 3 and 10 months, between 3 and 9 months, between 3 and 7 months, between 3 and 6 months, and between 3 and 4 months, with the most preferred range being between 3 and 4 months, and wherein said PCV2 ORF2 protein or immunological composition comprising a PCV2 ORF2 protein is to be administered two times to the piglets within an interval between administrations of 2 to 6 weeks, an interval between administrations of 2 to 5 weeks, an interval between administrations of 2 to 4 weeks, or an interval between administrations of 2 to 3 weeks. According to one aspect, the viral load in piglets older than 12 weeks of age, preferably in piglets between 12 to 20 weeks of age, in a herd of swine is reduced.

In another aspect of the invention, PCV2 ORF2 protein or an immunological composition comprising a PCV2 ORF2 protein is provided for use in a method for the reduction of the viral load, wherein said PCV2 ORF2 protein or immunological composition comprising a PCV2 ORF2 protein is to be administered at regular intervals to female animals of a herd and to piglets, wherein said regular intervals for the female animals are between 3 and 12 months, between 3 and 10 months, between 3 and 9 months, between 3 and 7 months, between 3 and 6 months, and between 3 and 4 months, with the most preferred range being between 3 and 4 months, and wherein said PCV2 ORF2 protein or immunological composition comprising a PCV2 ORF2 protein is to be administered two times to the piglets within an interval between administrations of 2 to 3 weeks. According to one aspect, the viral load in piglets older than 12 weeks of age, preferably in piglets between 12 to 20 weeks of age, in a herd of swine is reduced.

In another aspect of the invention, PCV2 ORF2 protein or an immunological composition comprising a PCV2 ORF2 protein is provided for use in a method for the reduction of the viral load, wherein said PCV2 ORF2 protein or immunological composition comprising a PCV2 ORF2 protein is to be administered at regular intervals to female animals of a herd and to piglets, wherein said regular intervals for the female animals are between 3 and 12 months, between 3 and 10 months, between 3 and 9 months, between 3 and 7 months, between 3 and 6 months, and between 3 and 4 months, with the most preferred range being between 3 and 4 months, and wherein said PCV2 ORF2 protein or immunological composition comprising a PCV2 ORF2 protein is to be administered two times to the piglets within an interval between administrations of 2 weeks. According to one aspect, the viral load in piglets older than 12 weeks of age, preferably in piglets between 12 to 20 weeks of age, in a herd of swine is reduced.

In another aspect of the invention, PCV2 ORF2 protein or an immunological composition comprising a PCV2 ORF2 protein is provided for use in a method for the reduction of the infection pressure in a herd of swine caused by an infection with PCV2, wherein said PCV2 ORF2 protein or immunological composition comprising a PCV2 ORF2 protein is to be administered at regular intervals to female animals of a herd and to piglets, wherein said regular intervals for the female animals are between 3 and 12 months, between 3 and 10 months, between 3 and 9 months, between 3 and 7 months, between 3 and 6 months, and between 3 and 4 months, with the most preferred range being between 3 and 4 months, and wherein said PCV2 ORF2 protein is to be administered one or more times to the piglets of the herd and wherein said PCV2 ORF2 protein is to be administered two or more times to the piglets within an interval of 2 to 6 weeks, an interval of 2 to 5 weeks, an interval of 2 to 4 weeks, or an interval of 2 to 3 weeks.

In another aspect of the invention, PCV2 ORF2 protein or an immunological composition comprising a PCV2 ORF2 protein is provided for use in a method for the reduction of the viral load, for the reduction of the infection quota and/or for the reduction of the infection pressure in a herd of swine caused by an infection with PCV2, wherein said PCV2 ORF2 protein or immunological composition comprising a PCV2 ORF2 protein is to be administered to female animals of the herd at regular intervals of between 3 and 12 months, between 3 and 10 months, between 3 and 9 months, between 3 and 7 months, between 3 and 6 months, and between 3 and 4 months, and wherein said PCV2 ORF2 protein is also to be administered two or more times to the piglets within an interval between administrations of 2 to 6 weeks, an interval between administrations of 2 to 5 weeks, an interval between administrations of 2 to 4 weeks, or an interval between administrations of 2 to 3 weeks. According to one aspect, the viral load in piglets older than 12 weeks of age, preferably in piglets between 12 to 20 weeks of age, in a herd of swine is reduced.

In another aspect of the invention, PCV2 ORF2 protein or an immunological composition comprising a PCV2 ORF2 protein is provided for use in a method for the reduction of the viral load, for the reduction of the infection quota and/or for the reduction of the infection pressure in a herd of swine caused by an infection with PCV2, wherein said PCV2 ORF2 protein or immunological composition comprising a PCV2 ORF2 protein is to be administered at regular intervals of 4 months to female animals of a herd and to piglets, wherein said PCV2 ORF2 protein or immunological composition comprising PCV2 ORF2 protein is to be administered one or more times to the piglets of the herd and wherein said PCV2 ORF2 protein is to be administered two or more times to the piglets within an interval of 2 to 6 weeks, an interval of 2 to 5 weeks, an interval of 2 to 4 weeks, or an interval of 2 to 3 weeks. According to one aspect, the viral load in piglets older than 12 weeks of age, preferably in piglets between 12 to 20 weeks of age, in a herd of swine is reduced.

In another aspect of the invention, PCV2 ORF2 protein or an immunological composition comprising a PCV2 ORF2 protein is provided for use according to any of the aspects herein, wherein said PCV2 ORF2 protein or immunological composition comprising a PCV2 ORF2 protein is to be administered to the piglets starting at 1 day of age.

In another aspect of the invention, the PCV2 ORF2 protein or immunological composition comprising a PCV2 ORF2 protein is administered to the piglets between day 1 and week 7 of age, preferably between day 1 and week 5 of age, and more preferably between day 1 and week 4 of age.

In another aspect of the invention, a PCV2 ORF2 protein or an immunological composition comprising a PCV2 ORF2 protein is provided for use according to any of the aspects herein, wherein said PCV2 ORF2 protein or immunological composition comprising a PCV2 ORF2 protein is to be administered to the piglets starting at 2 weeks of age.

In another aspect of the invention, a PCV2 ORF2 protein or an immunological composition comprising a PCV2 ORF2 protein is provided for use according to any of the aspects herein, wherein said PCV2 ORF2 protein or immunological composition comprising a PCV2 ORF2 protein is to be administered only one time to the piglets of the herd.

In another aspect of the invention, a PCV2 ORF2 protein or an immunological composition comprising a PCV2 ORF2 protein for use according to any of the aspects herein, wherein said PCV2 ORF2 protein or immunological composition comprising a PCV2 ORF2 protein is to be administered to the piglets only one time starting at 1 day of age. In other aspects, the PCV2 ORF2 protein or immunological composition comprising a PCV2 ORF2 protein is administered a single time to the piglets between day 1 and week 7 of age, preferably between day 1 and week 5 of age, and more preferably between day 1 and week 4 of age.

In another aspect of the invention, a PCV2 ORF2 protein or an immunological composition comprising a PCV2 ORF2 protein is provided for use according to any of the aspects herein, wherein said PCV2 ORF2 protein or immunological composition comprising a PCV2 ORF2 protein is to be administered to the piglets only one time starting at 2 weeks of age.

In another aspect of the invention, PCV2 ORF2 protein or an immunological composition comprising a PCV2 ORF2 protein is provided for use according to any of the aspects herein, wherein the PCV2 ORF2 protein or immunological composition comprising a PCV2 ORF2 protein is to be administered to 50% to 100% of the piglets in the herd, preferably to 60% to 100% of the piglets in the herd, even more preferably to 70% to 100% of the piglets in the herd, still more preferably to 80% to 100% of the piglets in the herd, and even more preferably administered to 90% to 100% of the piglets in the herd.

In another aspect of the invention, a PCV2 ORF2 protein or an immunological composition comprising a PCV2 ORF2 protein is provided for use according to any of the aspects herein, wherein said PCV2 ORF2 protein or immunological composition comprising a PCV2 ORF2 protein is to be administered to all piglets of a herd.

In another aspect of the invention, a PCV2 ORF2 protein or an immunological composition comprising a PCV2 ORF2 protein is provided for use according to any of the aspects herein, which prevents or reduces the clinical signs caused by a PCV2 infection in an animal within such herd.

In another aspect of the invention, the PCV2 ORF2 protein or immunological composition comprising a PCV2 ORF2 protein for use according to any of the aspects herein is administered to the piglets and/or to the female animals in a dose volume of up to 5 ml per dose, more preferably up to 4 ml per dose, still more preferably up to 3 ml per dose, more preferably up to 2 ml per dose, still more preferably from about 1 ml to 2 ml per dose, and most preferably about 1 ml per dose.

In another aspect of the invention, the PCV2 ORF2 protein or immunological composition comprising a PCV2 ORF2 protein for use according to any of the aspects herein can include one or more pharmaceutical-acceptable carriers, in particular an adjuvant.

In another aspect of the invention, the PCV2 ORF2 protein or immunological composition comprising PCV2 ORF2 protein may be administered along with an adjuvant or may be administered with no adjuvant at all. In an aspect where an adjuvant is included, preferred adjuvants include a polymer of acrylic or methacrylic acid, Aluminum hydroxide (ALOH), EMULSIGEN®, or Freud's Incomplete (oil) adjuvant, however, any adjuvant described herein will work for purposes of the present invention. In a further aspect, the PCV2 ORF2 protein or immunological composition comprising PCV2 ORF2 protein may be administered without an adjuvant.

In another aspect of the invention, the PCV2 ORF2 protein or immunological composition comprising PCV2 ORF2 protein comprises one further component selected from the group consisting of inactivated viral vector, cell culture supernate, media, viral inactivators, neutralization agents for viral inactivators, antibiotics, and combinations thereof.

In another aspect of the invention, the reduction of PCV2 viral load is accomplished in a herd by administration of PCV2 ORF2 protein or an immunogenic composition comprising PCV2 ORF2 protein to at least 50%, more preferably to at least 60%, even more preferably to at least 70%, still more preferably to at least 80%, even more preferably to at least 90%, and most preferably to at least 100% of the swine in the herd. The viral load is preferably reduced by at least 10%, more preferably by at least 20%, more preferably by at least 30%, still more preferably by at least 40%, even more preferably by at least 50%, still more preferably by at least 60%, more preferably by at least 70%, more preferably by at least 80%, still more preferably by at least 90%, and most preferably 100% reduction in the herd in comparison to the viral load in a herd that has not received an administration of PCV2 ORF2 protein or immunological composition comprising a PCV2 ORF2 protein. According to one aspect, the viral load in piglets older than 12 weeks of age, preferably in piglets between 12 to 20 weeks of age, in a herd of swine is reduced as described hereinabove.

In another aspect of the invention, the reduction of PCV2 viral titer is accomplished in a herd by administration of PCV2 ORF2 protein or an immunogenic composition comprising PCV2 ORF2 protein to at least 50%, more preferably to at least 60%, even more preferably to at least 70%, still more preferably to at least 80%, even more preferably to at least 90%, and most preferably to at least 100% of the swine in the herd. The viral titer of PCV2 is preferably reduced by at least 10%, more preferably by at least 20%, more preferably by at least 30%, still more preferably by at least 40%, even more preferably by at least 50%, still more preferably by at least 60%, more preferably by at least 70%, more preferably by at least 80%, still more preferably by at least 90%, and preferably by 100% in comparison to a herd that has not received an administration of the PCV2 ORF2 protein or immunological composition comprising PCV2 ORF2 protein. According to one aspect, the viral load in piglets older than 12 weeks of age, preferably in piglets between 12 to 20 weeks of age, in a herd of swine is reduced as described hereinabove.

In another aspect of the invention the number of animals in a herd that seroconvert is reduced by administration of PCV2 ORF2 protein or an immunogenic composition comprising PCV2 ORF2 protein. The viral load is preferably reduced by at least 10%, more preferably by at least 20%, more preferably by at least 30%, still more preferably by at least 40%, even more preferably by at least 50%, still more preferably by at least 60%, more preferably by at least 70%, more preferably by at least 80%, still more preferably by at least 90%, and preferably by 100% in comparison to the number of animals that did not receive an administration of PCV2 ORF2 protein or immunological composition comprising PCV2 ORF2 protein.

In another aspect of the invention, the percentage of PCV2 positive animals in a herd is reduced by the administration of PCV2 ORF2 protein or an immunogenic composition comprising PCV2 ORF2 protein. Preferably, the percentage of PCV2 positive animals in a herd is reduced by at least 10%, more preferably by at least 20%, more preferably by at least 30%, still more preferably by at least 40%, even more preferably by at least 50%, still more preferably by at least 60%, more preferably by at least 70%, more preferably by at least 80%, still more preferably by at least 90%, and preferably by 100% in comparison to the percentage of PCV2 positive animals in a herd that did not receive an administration of PCV2 ORF2 protein or an immunological composition comprising PCV2 ORF2 protein. PCV2 positive animals are preferably determined by a PCR assay. According to one aspect, the percentage of positive piglets older than 12 weeks of age, preferably of piglets between 12 to 20 weeks of age, in a herd of swine is reduced as described hereinabove.

### EXAMPLES

The main objective of the present study was to evaluate the feasibility to reduce PCV2 in a conventional farm by vaccinating both sows and piglets. Moreover, this study provided very useful information in terms of response after vaccination in a sow and piglet population. Therefore, a specific objective to be met within this general one was to study the humoral immunological response after vaccination in sows and piglets in different batches of the same farm. The deliverable of this project would be the proof of concept about the feasibility to reduce PCV2 by means of mass vaccination.

A field study will be carried out to assess the feasibility of PCV2 reduction in a conventional farm by means of vaccinating both sows and piglets. Details on the materials and methods and experimental design are given below.

### Farm and Vaccine

A conventional multi-site farm not suffering from PMWS, with confirmed PCV2 infection, was used. This farm supplied with a unique origin without mixing animals of different ages. The PCV2 vaccine was a subunit vaccine containing a PCV2 protein expressed in a baculovirus system.

### Study Design

### Vaccination Program

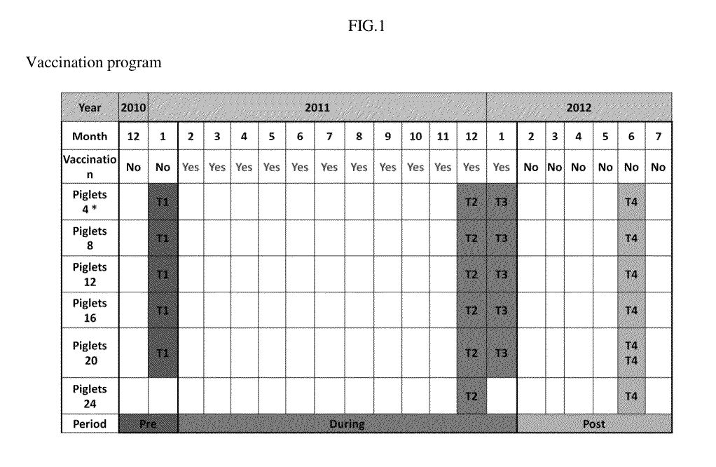

The study design is shown in FIG. 1. The vaccination strategy was similar to the one used in Aujeszky's disease reduction programs elsewhere (Pensaert, M and Morrison, R B, 2000. Vet Res. January-February; 31(1):141-5). Therefore, all the sows of the farm were vaccinated every three to four months (three to four doses of vaccine per sow during a year) and piglets will be vaccinated twice at 4 and 7 weeks of age. In order to avoid putative maternal immunity interference in piglet coming from vaccinated sows, the first dose of piglet vaccination was administered later (4 weeks) than the usually recommended age of vaccination (3 weeks).

This vaccination strategy was applied during 12 consecutive months. However, and in order to know the PCV2 status of the piglet and sow population before the vaccination, two consecutives groups of sows and piglets will be not vaccinated. Therefore, vaccination strategy will start at the 3rd group of sows and piglets. Vaccination program is shown in FIG. 1.

### Samplings

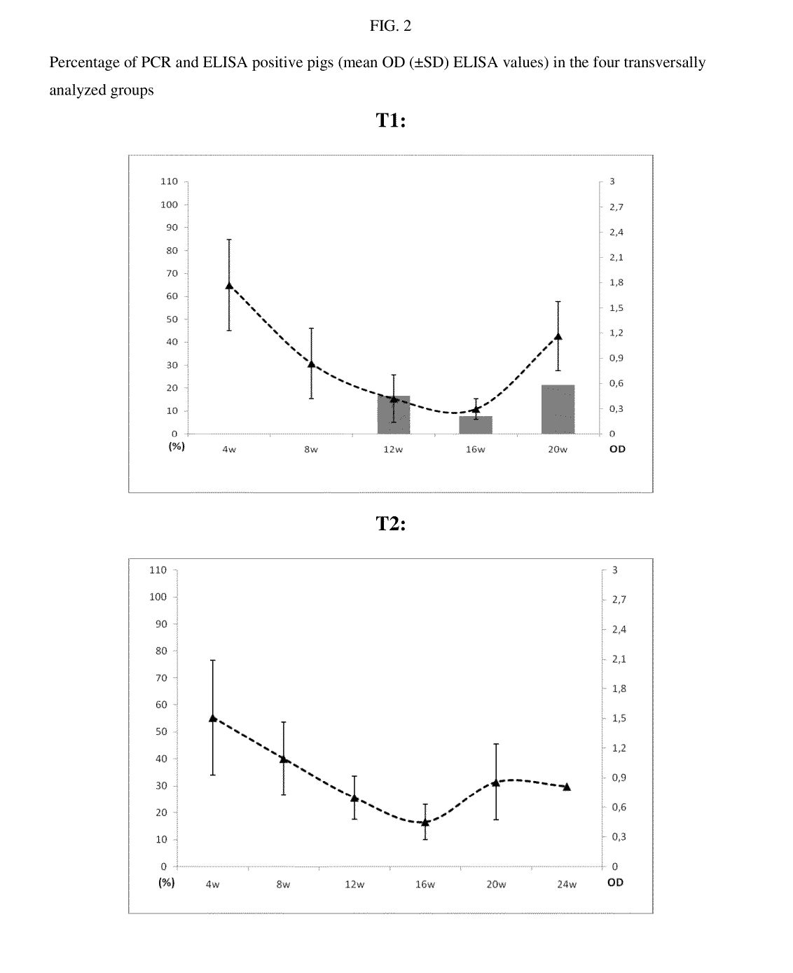

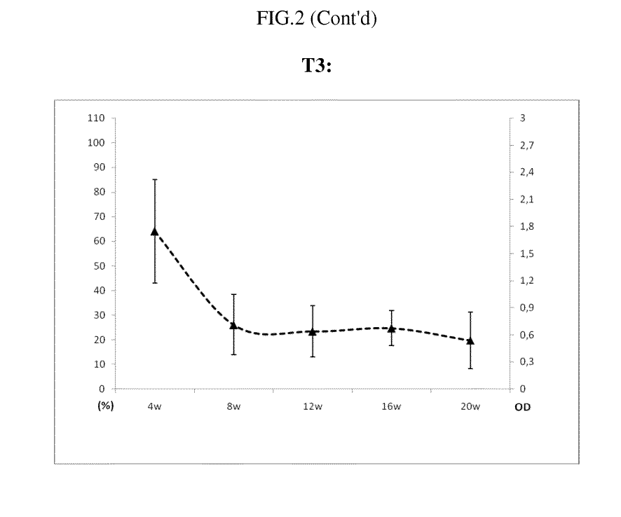

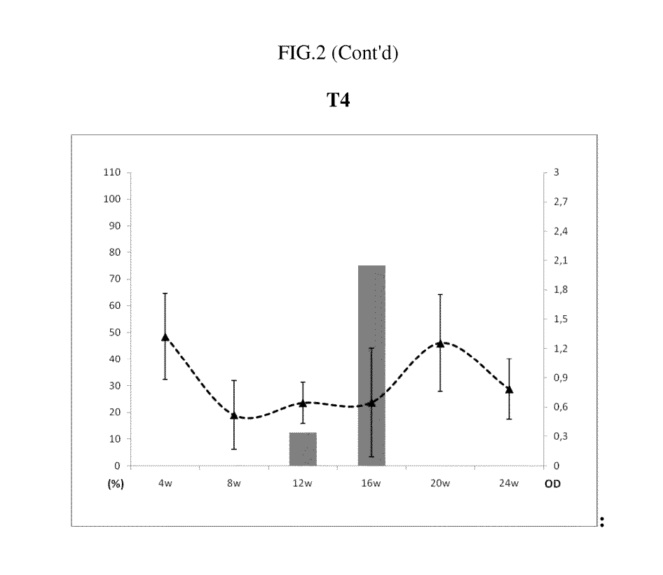

Number of samples taken is represented in FIG. 2. This experimental design implies a transversal as well as a longitudinal study at the same time. Blood samples from 15 (5 gilts, 5 from 2nd to 5th parity and 5 older than 5th parity, respectively) sows will be taken every 4 weeks. On the other hand, blood samples from 15 ear-tagged piglets of 4, 8, 12, 16, 20 and 24 weeks of age will be monthly taken. This study design implies that the total follow-up was last for fourteen months. Treatments, housing, husbandry and slaughtering conditions was conform to the European Union Guidelines and Good Clinical Practices and will be identical for both study groups.

### Laboratorial Tests

Sera obtained from blood samples will be analyzed by means of immunoperoxidasa assay (IPMA) and PCV2 PCR (Fort, M et al., 2008. Vaccine 26, 1063-1071). Those PCV2 PCR positive animals will subsequently be tested by a quantitative PCR (Olvera, A et al., 2004. J Virol Methods 117, 75-80).

### Remarks

All boars present in the farm were included in the vaccination program. Moreover, new entries (gilts, boars or piglets) was vaccinated and revaccinated during the quarantine and acclimatization period and afterwards included in the vaccination program.

### Results

No evidence of infection (no significant seroconversion and negative PCR results) in 2 consecutive transversal (T2 and T3) was observed. After termination of the vaccination regimen, a significant increase in number of PCR positive piglets as well as in seroconverted piglets was observed.

1. A method for the reduction of the viral load, reduction of the infection quota and/or the reduction of the infection pressure in a herd of swine caused by an infection with PCV2, comprising administering PCV2 ORF2 protein or an immunological composition comprising a PCV2 ORF2 protein, at regular intervals to female animals of a herd and to piglets.

2. The method of claim 1 for the reduction of the viral load in a herd of swine caused by an infection with PCV2.

3. The method of claim 1 for the reduction of the infection quota in a herd of swine caused by an infection with PCV2.

4. The method of claim 1 for the reduction of the infection pressure in a herd of swine caused by an infection with PCV2.

5. The method of claim 1, wherein said regular intervals are between 3 and 6 months.

6. The method of claim 1, wherein the regular interval is about 4 months.

7. The method of claim 1, wherein said PCV2 ORF2 protein or immunological composition comprising a PCV2 ORF2 protein is administered during the whole lifetime of the female animals.

8. The method of claim 1, wherein said PCV2 ORF2 protein or immunological composition comprising a PCV2 ORF2 protein is administered to 50% to 100% of the female swine animals in the herd.

9. The method of claim 1, wherein said PCV2 ORF2 protein or immunological composition comprising a PCV2 ORF2 protein is administered to all of the female swine animals in the herd.

10. The method of claim 1, wherein said PCV2 ORF2 protein or immunological composition comprising a PCV2 ORF2 protein is administered one or more times to piglets of the herd.

11. The method of claim 10, wherein said PCV2 ORF2 protein or immunological composition comprising a PCV2 ORF2 protein is administered two times or more to the piglets within an interval of 2 to 6 weeks.

12. The method of claim 1, wherein said PCV2 ORF2 protein or immunological composition comprising a PCV2 ORF2 protein is administered to the piglets starting at 1 day of age.

13. The method of claim 1, wherein said PCV2 ORF2 protein or immunological composition comprising a PCV2 ORF2 protein is administered to the piglets starting at 2 weeks of age.

14. The method of claim 10, wherein said PCV2 ORF2 protein or immunological composition comprising a PCV2 ORF2 protein is administered at 4 weeks and 7 weeks of age to the piglets of the herd.

15. The method of claim 1, wherein said PCV2 ORF2 protein or immunological composition comprising a PCV2 ORF2 protein is administered only one time to the piglets of the herd.

16. The method of claim 15, wherein said PCV2 ORF2 protein or immunological composition comprising a PCV2 ORF2 protein is administered to the piglets starting at 1 day of age.

17. The method of claim 15, wherein said PCV2 ORF2 protein or immunological composition comprising a PCV2 ORF2 protein is administered to the piglets starting at 2 weeks of age.

18. The method of claim 1, wherein said PCV2 ORF2 protein or immunological composition comprising a PCV2 ORF2 protein is administered to all piglets of a herd.

19. The method of claim 1, wherein said administration of said PCV2 ORF2 protein or immunological composition comprising a PCV2 ORF2 protein prevents or reduces the clinical signs caused by a PCV2 infection in an animal within such herd.
